(19) United States
(12) Patent Application Publication
(10) Pub. No.: US 2017/0000810 A1
(43) Pub. Date: Jan. 5, 2017

(54) NUTRITIONAL COMPOSITION TO REDUCE METABOLIC STRESS IN INFANTS

(71) Applicant: NESTEC S.A., Vevey (CH)

(72) Inventors: Jalil Benyacoub, Epalínges (CH); Nanda De Groot, Pully (CH); Philippe Steenhout, La Tour-de-Peìlz (CH)

(73) Assignee: NESTEC S.A., Vevey (CH)

(21) Appl. No.: 15/106,427

(22) PCT Filed: Dec. 18, 2014
(86) PCT No.: PCT/EP2014/078451

§ 371 Date: Jun. 20, 2016

(30) Foreign Application Priority Data
Dec. 19, 2013 (EP) 13198337.1

(51) Int. Cl. A61K 31/702; A23L 33/135; A23L 33/00; A23L 33/21; A61K 9/00; A61K 35/741

(52) U.S. Cl. CPC A61K 31/702; A61K 9/0053; A61K 35/741; A23L 33/40; A23L 33/21; A23L 33/135; A61K 2035/115

(57) ABSTRACT

The present invention relates to a nutritional composition comprising an oligosaccharide mixture, said oligosaccharide mixture comprising at least one N-acetylated oligosaccharide, one galacto-oligosaccharide and one sialylated oligosaccharide for use in reducing the metabolic stress in an infant in the first twelve months of life and/or decreasing gut permeability in an infant in the first twelve months of life and/or promoting a rate of growth in an infant fed with said nutritional composition in the first twelve months of life which approximates to the rate of growth of a breast-fed infant at the same age.

### FIELD OF THE INVENTION

The invention relates to a nutritional composition such as an infant formula, comprising an oligosaccharide mixture which is specifically designed to reduce the metabolic stress, decrease the gut permeability and promote the rate of growth of an infant, especially in the first twelve months of life.

### BACKGROUND OF THE INVENTION

The human colon is colonized with a wide range of bacteria that have either beneficial or harmful effects on gut physiology as well as having other systemic influences. Predominant groups of bacteria found in the colon include bacteroides, bifidobacteria, eubacteria, clostridia and lactobacilli. The bacteria present have fluctuating activities in the response to substrate availability, redox potential, pH, O2 tension and distribution in the colon. Pathogenic effects (which may be caused by clostridia or bacteroides, for example) include diarrhoea, infections, liver damage, carcinogenesis and intestinal putrefaction. Health-promoting effects may be caused by inhibition of growth of, and colonization by, harmful bacteria, stimulation of the immune functions, improving digestion and absorption of essential nutrients and synthesis of vitamins.

At birth, the gastro-intestinal tract of an infant is thought to be sterile. During the process of birth, it encounters bacteria from the digestive tract and skin of the mother and starts to become colonized. Large differences exist with respect to the composition of the gut microbiota in response to infant feeding.

Mother's milk is recommended for all infants. However, in some cases breast feeding is inadequate or unsuccessful for medical reasons or mother chooses not to breast feed. Infant formulas have been developed for these situations. The use of probiotics has therefore been investigated in the past and several infant formulas have been supplemented with probiotic bacterial strains.

As the composition of human milk becomes better understood, it has also been proposed to add prebiotics to infant formula. Various infant formulas supplemented with prebiotics such as mixtures of fructo-oligosaccharides (FOS) and galacto-oligosaccharides (GOS) for example are commercially available. Prebiotics are non-digestible in the sense that they are not broken down and absorbed in the stomach or small intestine and thus pass intact to the colon where they are selectively fermented by the bacteria. The main effect of prebiotics, once fermented, is to selectively promote the growth and metabolic activity of certain species of bacteria recognized as beneficial for the host well-being and health (Roberfroid, M, J. Nutrition, 2007: 37(3): 830S-837S). Beyond the direct effects of prebiotic on the gastrointestinal flora, prebiotics are known to also have beneficial effects on the host health (such as anticarcinogenic effects, improvement of mineral absorption and effects on metabolite production) that may be due to indirect effects of the prebiotic on the gut microflora.

However, commercially available mixtures approximate only roughly the mixture of oligosaccharides found in human milk. More than 120 different oligosaccharide components have been detected in human milk, some of which have not been detected so far in animal milks (such as bovine milk) at all or have been detected only in small quantities. Some classes of human milk oligosaccharides are present in bovine milk or colostrum only in very small quantities or not at all are sialylated and fucosylated oligosaccharides. As bovine milk contains some oligosaccharides that are structurally identical or similar to those found in human milk, oligosaccharides from bovine milk in sufficient quantities should have prebiotic effect or other beneficial properties associated with human milk oligosaccharides. However until recently, the low concentration of these oligosaccharides in bovine milk (about 20-fold lower than in human milk) has hampered efforts to utilize bovine milk as a source of oligosaccharides for infant formulas.

Human milk is known to contain a larger amount of ingestible oligosaccharides than most other animal milks. In fact, ingestible oligosaccharides represent the third largest solid component (after lactose and lipids) in breast milk, present at a concentration of 12-15 g/l in colostrum and 5-8 g/l in mature milk. Human milk oligosaccharides are very resistant to enzymatic hydrolysis, indicating that these oligosaccharides may display essential functions not directly related to their calorific value.

Infant formulas have been developed to provide a composition that could beneficially substitute for human milk. The patent application US2003/0129278 describes an oligosaccharide mixture based on oligosaccharides produced from one or several animal milks which is characterized in that it comprises at least two oligosaccharide fractions which are each composed of at least two different oligosaccharides, with free lactose not pertaining thereto. The total spectrum of oligosaccharides present in the oligosaccharide mixture differs from those present in the animal milk or animal milks from which the oligosaccharide fractions were extracted. Further a) if said oligosaccharides are extracted from only one animal milk, the proportion of neutral oligosaccharides to acidic sialylated oligosaccharides is 90-60:10-40 weight %, or b) if said oligosaccharides are extracted from at least two animal milks, the oligosaccharides extracted from different animal milks each make up 10 weight % of the total amount of oligosaccharides present in the oligosaccharide mixture.

It has been widely reported that breast fed infants do have a different growth pattern than infants fed with infant formula. Indeed, infant fed with infant formula have a lower weight gain and a lower body fat mass within the first year of life as compared to breast fed infants. Additionally, breast fed infant have a different gut microbiota profile as compared to infant fed with infant formula. Altogether, these factors affect the development of the infant physiology, including metabolism, immunity and overall growth.

The patent application WO2007/090894 describes an oligosaccharide mixture which comprises 5 to 70 weight % of at least one N-acetylated oligosaccharide, 20 to 90 weight % of at least one neutral galacto-oligosaccharide and 5 to 50 weight % of at least one sialylated oligosaccharide. Said oligosaccharide mixture is described as having an effect especially on the establishment and composition of the intestinal microbiota in infants.

The patent application WO2007/101675 describes a preparation that comprises a probiotic bacterial strain and a prebiotic mixture comprising 5-70 weight % of at least one N-acetylated oligosaccharide, 20-95 weight % of at least one neutral oligosaccharide and 2-50 weight % of at least one sialylated oligosaccharide. Said preparation is used in the prevention and treatment of infections.

The patent application WO2010/003803 describes a nutritional composition for administration to infants which comprises 2.5 to 15.0 weight % of an oligosaccharide mixture consisting of N-acetylated oligosaccharide(s), galacto-oligosaccharide(s) and sialylated oligosaccharide(s). Said composition is administered to an infant in the first six months of life to reduce the risk of obesity later in life.

However, none of these prior art documents addresses the issue of reducing the metabolic stress in infants that may happen for example by the introduction of infant formula, and that may affect the gut permeability. There is also no focus on providing a nutritional composition which promotes a rate of growth of the infant that is closer to the one obtained for breast-fed infants.

There is therefore a need to develop specific compositions suitable to reduce the metabolic stress and/or suitable to decrease the gastrointestinal permeability and/or suitable to promote a proper rate of growth in an infant in the first twelve months of life.

There is also a need to deliver such health benefits in a manner that is particularly adapted for these young subjects, in a manner that does not involve a classical pharmaceutical intervention as infants are particularly fragile.

There is a need to deliver such health benefits in these young subjects in a manner that does not induce side effects and/or in a manner that is easy of deliver, and well accepted by the parents or health care practitioners.

There is also a need to deliver such benefits in a manner that does keep the cost of such delivery reasonable and affordable by most.

### SUMMARY OF THE INVENTION

It has been surprisingly found that a nutritional composition which comprises a specific mixture of bovine's milk oligosaccharides (BMOs) reduces the metabolic stress, decreases the gut permeability and improves the rate of growth of infants fed with the described nutritional composition as compared to infants fed with conventional nutritional compositions in order to get a profile closer to the one obtained for breast-fed infants.

Accordingly, the present invention provides a nutritional composition comprising an oligosaccharide mixture, said oligosaccharide mixture comprising at least one N-acetylated oligosaccharide, one galacto-oligosaccharide and one sialylated oligosaccharide for use in reducing the metabolic stress (and therefore reducing the metabolic disorders and/or imbalances) in an infant in the first twelve months of life.

Another object of the invention refers to a nutritional composition comprising an oligosaccharide mixture, said oligosaccharide mixture comprising at least one N-acetylated oligosaccharide, one galacto-oligosaccharide and one sialylated oligosaccharide for use in decreasing gut permeability in an infant in the first twelve months of life.

In a third aspect, the invention provides a nutritional composition comprising an oligosaccharide mixture, said oligosaccharide mixture comprising at least one N-acetylated oligosaccharide, one galacto-oligosaccharide and one sialylated oligosaccharide for use in promoting a rate of growth in the first twelve months of life in an infant fed with said nutritional composition which approximates to the rate of growth of a breast-fed infant at the same age.

In a particular embodiment, the nutritional composition comprises from 2.5 to 15.0 wt % of the oligosaccharide mixture.

In another embodiment, the nutritional composition comprises at least 0.01 wt % of N-acetylated oligosaccharide(s), at least 2.0 wt % of galacto-oligosaccharide(s) and at least 0.02 wt % of sialylated oligosaccharide(s).

In one embodiment, the oligosaccharide mixture comprises from 0.1 to 4.0 wt % of the N-acetylated oligosaccharide(s), from 92.0 to 98.5 wt % of the galacto-oligosaccharide(s) and from 0.3 to 4.0 wt % of the sialylated oligosaccharide(s).

In one embodiment the oligosaccharide mixture is derived from animal milk, such as cow's milk, goat's milk or buffalo's milk.

In another aspect, the invention relates is an infant formula which can be administered to the infant within the first twelve months of life, such as within the first month of life.

### BRIEF DESCRIPTION OF THE FIGURES

FIG. 1 shows the levels of fecal Elastase measured during the first 4 weeks of life of infants fed either with a standard infant formula, or with a standard infant formula supplemented with bovine milk oligosaccharides or in breast-fed infants.

FIG. 2 shows the levels of fecal α1-Antitrypsin measured during the first 4 weeks of life of infants fed either with a standard infant formula, or with a standard infant formula supplemented with bovine milk oligosaccharides or in breast-fed infants.

FIG. 3 shows the evolution of the weight during the first 8 weeks of life of infants fed either with a standard infant formula, or with a standard infant formula supplemented with bovine milk oligosaccharides or in breast-fed infants.

FIG. 4 shows the evolution of the height during the first 8 weeks of life of infants fed either with a standard infant formula, or with a standard infant formula supplemented with bovine milk oligosaccharides or in breast-fed infants.

### DETAILED DESCRIPTION OF SOME EMBODIMENTS OF THE INVENTION

In this specification, the following terms or expressions are given a definition that must be taken into account when reading and interpreting the description, examples and claims.

The term “infant” means a child until the age of 12 months. In the context of the present invention, the infant may be any term infant or preterm infant. Infant may have been delivered either by vaginal delivery (also referred to as natural delivery) or caesarean section (also referred to as C-section). In some embodiments of the invention, the infant is a term infant.

The expression “nutritional composition” means a composition which nourishes a subject. This nutritional composition is usually to be taken orally or intravenously, and it usually includes a lipid or fat source, a carbohydrate source and a protein source.

In the context of the present invention, the nutritional compositions are typically “synthetic nutritional compositions”, i.e. not of human origin (e.g. this is not breast milk). The expression “synthetic nutritional composition” means a mixture obtained by chemical and/or biological means, which can be chemically identical to the mixture naturally occurring in mammalian milks.

In some embodiments of the invention, the nutritional composition is a hypoallergenic nutritional composition. The expression “hypoallergenic nutritional composition” means a nutritional composition which is unlikely to cause allergic reactions.

The nutritional compositions according to the invention may be for example an infant formula, any other milk-based nutritional composition, a supplement (or a complement), a fortifier such as a milk fortifier. The nutritional compositions can be in powder or liquid form.

The expression “infant formula” means a foodstuff intended for the complete nutrition of infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” includes the starter formulas and the hypoallergenic infant formulas.

The term “oligosaccharide” means a carbohydrate having a degree of polymerization (DP) ranging from 2 to 20 inclusive but not including lactose. In some embodiments of the invention, carbohydrate has DP ranging from 3 to 20.

The expressions “oligosaccharide mixture” or “mixture of oligosaccharide” can be used interchangeably. The “oligosaccharide mixture” according to the invention comprises at least one N-acetylated oligosaccharide, at least one galacto-oligosaccharide and at least one sialylated oligosaccharide. The mixture may be made of one or several oligosaccharides of these different types, i.e. one or several N-acetylated oligosaccharide(s), one or several galacto-oligosaccharide(s) and one or several sialylated oligosaccharide(s). In some advantageous embodiments the oligosaccharides of the oligosaccharide mixture are bovine's milk oligosaccharides (or BMOs).

The expression “N-acetylated oligosaccharide” means an oligosaccharide having N-acetyl residue.

The expressions “galacto-oligosaccharide” and “GOS” can be used interchangeably. They refer to an oligosaccharide comprising two or more galactose molecules which has no charge and no N-acetyl residue (i.e. they are neutral oligosaccharide). In a particular embodiment, said two or more galactose molecules are linked by a β-1,2, β-1,3, β-1,4 or β-1,6 linkage.

In another embodiment, “galacto-oligosaccharide” and “GOS” also include oligosaccharides comprising one galactose molecule and one glucose molecule (i.e. disaccharides) which are linked by a β-1,2, β-1,3 or β-1,6 linkage.

The expression “sialylated oligosaccharide” means an oligosaccharide having a sialic acid residue with associated charge.

The term “prebiotic” means a non-digestible food ingredient that beneficially affects the host by selectively stimulating the growth and/or activity of one or a limited number of bacteria in the colon and thus improves host health (Gibson and Roberfroid “Dietary Modulation of the Human Colonic Microbiota: Introducing the Concept of Prebiotics”, J. Nutr. 1995: 125(6):1401-1412). “Prebiotics” alternatively means selectively fermented ingredients that allow specific changes, both in the composition and/or activity in the gastrointestinal microflora, that confer benefits upon the host well-being and health (Roberfroid M. “Prebiotics: the concept revisited”, J. Nutr. 2007: 37 (3): 830S-837S).

The term “probiotic” means microbial cell preparations or components of microbial cells with a beneficial effect on the health of the host (Salminen S. Ouwehand A, Benno Y. et al. “Probiotics: how should they be defined” Trend Food Sci. Technol. 1999: 10: 107-10). The microbial cells are generally bacteria or yeasts. In some particular embodiments the probiotic is a probiotic bacterial strain.

The term “cfu” should be understood as colony-forming unit.

The expression “metabolic stress” should be understood as a situation during which an unforeseen physical, chemical or biological factor (insult) brutally modifies homeostasis, therefore nutrient's metabolism and nutritional needs of an individual (Colomb, V., Nutrition Clinique et Métabolisme, 2005: 19: 229-33). In the context of the present invention, the stress factor considered in this case (i.e. in infant) may be mainly due to the change in feeding and the introduction of an infant formula containing substances that are encountered for the first time by the infant organism. The way of delivery may also be considered as a stress factor. C-section may induce stress that impacts metabolic health in the newborn. A too frequent antibiotic use early in life may also be a factor inducing a metabolic stress, as well as the fact the infant is born preterm and/or small-for-gestational age. The expression “reducing the metabolic stress” of an individual implies a reduction of the metabolic disorders and/or imbalances—especially those resulting from an unforeseen chemical, nutritional or biological factor (insult)—such as a change of homeostasis, nutrient's metabolism, nutritional needs of said individual. It also encompasses the treatment (e.g. a reduction of the occurrences/severities) of conditions and/or diseases associated to the metabolic disorders and/or imbalances, known by the skilled person. One embodiment of the present invention therefore refers to a nutritional composition comprising an oligosaccharide mixture as described in the present invention for use in the prevention and/or treatment of conditions and/or diseases associated to the metabolic disorders and/or imbalances in an infant, especially by reducing the metabolic stress in said infant in the first twelve months of life. Some examples of conditions and/or diseases associated to the metabolic disorders and/or imbalances include neurological, growth and/or gut retarded development or abnormalities, hypoglycemia, hyperglycemia, hyperinsulinemia, hypertriglyceridemia.

The expressions “gut permeability” or “intestinal permeability” or “gastrointestinal permeability” designate the absorptive ability of the gut and can be defined as the capacity of the mucosal surface to be penetrated by specific substances through unmediated diffusion. As the gut permeability is closely linked with gut barrier function, decreasing gut permeability may allow strengthening the intestinal barrier and favouring a suitable metabolism in said infant.

The expressions “growth rate” and “rate of growth” can be used interchangeably. They refer to growth in weight, height and/or head circumference of an infant. The growth has to be understood as the evolution of the weight, height and/or head circumference over the aging of the infant. These parameters do not exclusively increase during development of the infant, as indeed the standard curves of growth published by the WHO show that the weight of an infant may decrease in the first days of life of the infant. Therefore, the rate of growth has to be understood as the overall growth of the infant over the first months of life.

The expression “rate of growth of a breast-fed infant” refers to the rate of growth of an infant who is not fed with a nutritional composition but who is exclusively breast-fed.

In the expression “promoting a rate of growth [ . . . ] in an infant fed with said nutritional composition which approximates to the rate of growth of a breast-fed infant at the same age”, the term “approximates” refers to a rate of growth which is closer to the one of breast-fed infants as compared to the rate of growth of an infant fed with a standard nutritional composition that does not comprises the oligosaccharide mixture of the invention. The term “approximates” does not mean that the rate of growth has to be equal to that of breast-fed infants, it just has to be closer to that of breast-fed infants, than the rate of growth of infants fed with standard nutritional composition are to the breast-fed infants. Said expression includes the prevention and/or treatment of growth rate abnormalities (e.g. growth retardation, too fast growth or excessive growth) that may occur in formula-fed infants. Some growth rate abnormalities may indeed often be observed in formula-fed infants as the introduction of an infant formula may increase the metabolic stress of said infant: the formula-fed infants may encounter a growth retardation during the first few weeks of life in comparison to breast-fed infants, then a period of too fast growth (catch-up growth) to compensate this growth delay, then an excess of growth in the subsequent months of life of the formula-fed infant, in comparison to breast-fed infants. These growth rate abnormalities will further increase the metabolic stress and the risks associated thereof in said infants (vicious circle). In the context of the present invention, the expression “promoting a rate of growth” in an infant fed with the nutritional composition of the invention generally may refer to promoting a more constant/normal growth rate (e.g. regulation of the growth rate speed) of said infant in comparison to breast-fed infants, e.g. by an increase of the rate of growth in the first few weeks of life of the infant (e.g. the first 2 weeks, the first 3 weeks, the first 4 weeks, the first 5 weeks, the first 6 weeks, the first 7 weeks or the first 8 weeks of life of the infant) and/or a decrease of its rate of growth in the subsequent months of life.

All percentages are by weight unless otherwise stated. The expressions “weight %” and “wt %” are synonymous. They refer to quantities expressed in percent on a dry weight basis.

It is noted that the various aspects, features, examples and embodiments described in the present application may be compatible and/or combined together.

In addition, in the context of the invention, the terms “comprising” or “comprises” do not exclude other possible elements. In some particular embodiments the terms “comprising” or “comprises” also encompass the expressions “consisting of”, “consists of”, “consisting essentially of”, “consists essentially of”.

The present inventors have surprisingly found that a nutritional composition comprising a particular mixture of oligosaccharides is particularly effective for reducing the metabolic stress and/or for decreasing the gut permeability in an infant in the first twelve months of life and/or for promoting a rate of growth in the first twelve months of life in an infant fed with said nutritional composition which approximates to the rate of growth of a breast-fed infant at the same age.

Metabolic stress affects the body of an individual in different ways, and it generally modifies homeostasis and therefore both nutrient's metabolism and nutritional needs. Metabolic stress is induced by an unforeseen physical, chemical or biological factor which brutally modifies the homeostasis and may lead to deficiencies of one or many organs in the worst cases.

The metabolic stress in infant may happen for example upon introduction of infant formula as it may contain substances and/or nutrients that were never encountered by the infant organism before. Therefore, it is known that infant fed with infant formula, rather than fed with breast milk, experience metabolic stress. Infant formula induced metabolic stress could play a part in the link between formula-feeding and an increased risk of obesity, type-2 diabetes and risk of chronic diseases later in life. Infant feeding with formula influence metabolism in developing infants and may be the link between early feeding and the development of metabolic diseases later in life (REF: O'Sullivan A, et al. Journal of Proteome Research: 2013: 12 (6): 2833-45).

Metabolic stress can be monitored by measuring the levels of different physiological markers in infant stool samples. For example, Elastase, an enzyme from pancreatic origin, is not degraded in the gut and therefore can be measured to evaluate the pancreatic activity as reflect of induced metabolism. Elevation in the level of Elastase reflects an increased metabolic activity. As illustrated in the example 2, it has been surprisingly observed by the present inventors that infants who received the nutritional composition of the present invention showed levels of Elastase comparable to the level of Elastase measured in breast-fed infants. In the contrary, infants fed with standard infant formula showed an elevated level of Elastase as compared to the level measured in breast fed infants. Without being bound by the theory, the present inventors believes that the nutritional composition of the present invention directly reduces metabolic stress, as reflected by the levels of fecal Elastase measured in infants fed with said nutritional composition.

Therefore, a first object of the invention is to provide a nutritional composition comprising an oligosaccharide mixture, said oligosaccharide mixture comprising at least one N-acetylated oligosaccharide, one galacto-oligosaccharide and one sialylated oligosaccharide for use in reducing the metabolic stress in an infant in the first twelve months of life.

The intestinal cells form a tight but selective barrier. Intestinal mucosa has an absorptive function (for nutrients for example) and acts also as a selective barrier retaining substances in the gut or preventing against potential toxic, antigenic or carcinogenic substances. The intestinal permeability is thought to happen via two hypothetical permeation routes: the first one hypothesizes a transcellular (through small pores), a paracellular (through big channels) and a lyophilic pathways; the 15 second one gives a key role to only paracellular tight-junctions. Absorptive capabilities improve during the maturation of the gut which generally happens within the first 6 months to 2 years following birth.

The gut epithelial integrity is itself dynamic and matures over time starting soon after birth, though the mechanisms regulating dynamic gut permeability are poorly understood.

The gut permeability is closely linked with the barrier function of the gut. If the intestinal barrier starts to become too permeable, it can cause many problems in the body. Indeed, high intestinal permeability may allow a foreign substance to penetrate the bloodstream and consequently the immune system is activated to eliminate or destroy this foreign molecule. The activation of the immune system leads to an inflammatory response. An increased inflammatory response increases the risk of all chronic diseases. Therefore, gut permeability will also influence the inflammatory load of a person.

Gut permeability can be monitored by the measure of α1-antitrypsin (AAT), a protein belonging to the family of serpins (serine protease inhibitor). AAT is used as a marker for intestinal protein loss reflecting an increased intestinal permeability. AAT has been found to be expressed by human mammary glands during lactation and it has been suggested that AAT is passed to the infant by the mother during breast feeding. It has been postulated that the milk-AAT might remain intact in neonatal gut and contributes to protect and increase survival of other protein milk via partial inhibition of pancreatic proteases.

It has been shown that AAT was a relevant marker for intestinal-inflammation associated diseases, and that measurement of fecal AAT in children with severe intestinal disorders is a reliable tool supporting the diagnosis of protein-loss enteropathy. Therefore, elevated levels of fecal AAT are an indication of intestinal inflammation and increased gut permeability in infants.

As illustrated in the example 2, the inventors have surprisingly found that the nutritional composition of the present invention improves the gut permeability as compared to standard infant formula. Indeed, infants fed with the nutritional composition of the present invention show a level of fecal AAT comparable to levels of AAT measured in breast-fed infants, while infant fed with standard infant formula have a higher level of fecal AAT compared to breast-fed infants. Therefore the gut permeability as observed in infants fed with the nutritional composition of the present invention approaches the gut permeability in breast-fed infants. Without being bound by the theory, the inventors believe that the oligosaccharides comprised in the nutritional composition acts on the gut permeability of the infants and may have a beneficial effect on the retention of substances, for example proteins, this effect on gut permeability not being necessarily linked to the gut microbiota.

Therefore the present invention also refers to a nutritional composition comprising an oligosaccharide mixture, said oligosaccharide mixture comprising at least one N-acetylated oligosaccharide, one galacto-oligosaccharide and one sialylated oligosaccharide for use in decreasing gut permeability in an infant in the first twelve months of life.

Growth monitoring of infants is the regular measurement of an infant's size in order to document growth. Growth monitoring is important as it can detect early changes in infant's growth. Both growing too slowly or too fast may indicate a nutritional or other health problem. Therefore, growth monitoring is an essential part of primary health care in infants. Growth is usually monitored by following the height, weight and head circumference of the infant over a certain period of time, for example over the first few weeks or months of life of said infants. Weight-for-age is particularly useful in small infants who normally gain weight fast. Normal weight gain suggests that the infant is healthy and growing normally. Failure to gain weight normally is of the earliest sign of illness or malnutrition. Height is also an important measure of linear growth (stature) as height reflects growth over a longer period that does weight. Head circumference can be used to assess brain growth in infants less than 2 years. During this period brain growth is fast and therefore head circumference increase rapidly. A small head (microcephaly) suggests a small brain, while a large head suggests hydrocephaly. The World Health Organisation (WHO) is releasing international growth standard statistical distribution which describes the growth of children ages 0 to 59 months living in environments believed to support optimal growth in 6 countries throughout the world. These standard statistical values are used as targeted value (Z-score) in the study illustrated in the example 2. Any significant deviation from the Z-Score indicates an abnormal growth.

As illustrated in the example 2, the inventors have found that infants fed with the nutritional composition according to the present invention have a rate of growth which approximates the rate of growth of breast-fed infants at the same age, this effect on rate of growth not being necessarily linked to the gut microbiota. Therefore, the nutritional composition comprising an oligosaccharide mixture promotes a rate of growth that approximates the rate of growth of a breast fed infant, i.e. normalizes the rate of growth of an infant fed with said nutritional composition. Importantly, the growth of the infants fed with the nutritional composition of the present invention approaches the targeted standard value (Z-Score) advised by the WHO.

In another aspect, the invention provides a nutritional composition comprising an oligosaccharide mixture, said oligosaccharide mixture comprising at least one N-acetylated oligosaccharide, one galacto-oligosaccharide and one sialylated oligosaccharide for use in promoting a rate of growth in the first twelve months of life in an infant fed with said nutritional composition which approximates to the rate of a breast-fed infant at the same age.

Without being bound by theory, the inventors of the present invention believe that the different oligosaccharides may act synergically, and are able to provide the above-mentioned effects beyond the positive effect on gut microbiota. Indeed, ingestion of oligosaccharides may also have pleiotropic effects outside the gastrointestinal tract, these effects not necessarily being linked to the gut microbiota. Systemic effects of oligosaccharides were reported to modulate for example hepatic metabolism, glucose metabolism or hormone metabolism (Dlezenne, Proc. Nutr. Soc. 2003: 62: 177-182).

The oligosaccharide mixture of the nutritional composition according to the invention comprises at least one N-acetylated oligosaccharide, at least one galacto-oligosaccharide and at least one sialylated oligosaccharide. As previously mentioned, there may be made of one or several oligosaccharides of these different types, i.e. one or several N-acetylated oligosaccharide(s), one or several galacto-oligosaccharide(s) and one or several sialylated oligosaccharide(s). The oligosaccharide mixture of the nutritional composition of the invention may be prepared from one or more animal milks. The milk may be obtained from any mammal, in particular from cows, goats, buffalos, horses, elephants, camels or sheep.

Alternatively the oligosaccharide mixture may be prepared by purchasing and mixing the individual components.

An N-acetylated oligosaccharide is an oligosaccharide having an N-acetylated residue. Suitable N-acetylated oligosaccharides of the oligosaccharide mixture of the nutritional composition according to the present invention include GalNAcα1,3Galβ1,4Glc and Galβ1,6GalNAcα1,3Galβ1,4Glc, but also any mixture thereof. The N-acetylated oligosaccharides may be prepared by the action of glucosaminidase and/or galactoaminidase on N-acetyl-glucose and/or N-acetyl galactose. Equally, N-acetyl-galactosyl transferases and/or N-acetyl-glycosyl transferases may be used for this purpose. The N-acetylated oligosaccharides may also be produced by fermentation technology using respective enzymes (recombinant or natural) and/or microbial fermentation. In the latter case the microbes may either express their natural enzymes and substrates or may be engineered to produce respective substrates and enzymes. Single microbial cultures or mixed cultures may be used. N-acetylated oligosaccharide formation can be initiated by acceptor substrates starting from any degree of polymerization (DP) from DP=1 onwards. Another option is the chemical conversion of keto-hexose (fructose) either free or bound to an oligosaccharide (e.g lactulose) into N-acetylhexosamine or an N-acetylhexosamine containing oligosaccharide as described in Wrodnigg, T. M, Dtutz, A. E, Angew. Chem. Int. Ed. 1999: 38: 827-828.

A galacto-oligosaccharide is an oligosaccharide comprising two or more galactose molecules which has no charge and no N-acetyl residue. Suitable galacto-oligosaccharides of the oligosaccharide mixture of the nutritional composition according to the present invention include Galβ1,3Galβ1,4Glc, Galβ1,6Galβ1,4Glc, Galβ1,3Galβ1,3Galβ1,4Glc, Galβ1,6Galβ1,6Galβ1,4Glc, Galβ1,3Galβ1,6Galβ1,4Glc, Galβ1,6Galβ1,3Galβ1,4Glc, Galβ1,6Galβ1,6Galβ1,6Glc, Galβ1,3Galβ1,3Glc, Galβ1,4Galβ1,4Glc and Galβ1,4Galβ1,4Galβ1,4Glc, but also any mixture thereof. Synthesized galacto-oligosaccharides such as Galβ1,6Galβ1,4Glc, Galβ1,6Galβ1,6Galβ1,6Glc, Galβ1,3Galβ1,4Glc, Galβ1,6Galβ1,6Galβ1,4Glc, Galβ1,6Galβ1,3Galβ1,4Glc, Galβ1,3Galβ1,6Galβ1,4Glc, Galβ1,4Galβ1,4Glc and Galβ1,4Galβ1,4Galβ1,4Glc and mixture thereof are commercially available under trademarks Vivinal® and Elix'or®. Other suppliers of oligosaccharides are Dextra Laboratories, Sigma-Aldrich Chemie GmbH and Kyowa Hakko Kogyo Co., Ltd. Alternatively, specific glycotransferases, such as galoctosyltransferases may be used to produce neutral oligosaccharides.

A sialylated oligosaccharide is an oligosaccharide having a sialic acid residue with associated charge. Suitable sialylated oligosaccharides of the oligosaccharide mixture of the nutritional composition according to the present invention include NeuAcα2,3Galβ1,4Glc and NeuAcα2,6Galβ1,4Glc, but also any mixture thereof. These sialylated oligosaccharides may be isolated by chromatographic or filtration technology from a natural source such as animal milks. Alternatively, they may also be produced by biotechnology using specific sialyltransferases either by enzyme based fermentation technology (recombinant or natural enzymes) or by microbial fermentation technology. In the latter case microbes may either express their natural enzymes and substrates or may be engineered to produce respective substrates and enzymes. Single microbial cultures or mixed cultures may be used. Sialyl-oligosaccharide formation can be initiated by acceptor substrates starting from any 25 degree of polymerization (DP) from DP=1 onwards.

In one aspect of the invention, the nutritional composition comprises the oligosaccharide mixture in an amount from 2.5 to 15 wt %. Alternatively, the nutritional composition comprises the oligosaccharide mixture in an amount from 3 to 15 wt %, or in an amount from 3 to 10 wt %, or in an amount from 3.5 to 9.5 wt % or in an amount from 4 to 9 wt % or in an amount from 4.5 to 8.5 wt %, or in an amount from 5.0 to 7.5 wt % or in an amount from 5 to 8 wt %.

In some specific embodiments, the nutritional composition may comprise the oligosaccharide mixture in an amount from 0.5 to 3.1 g/100 kcal, or in an amount from 0.6 to 3.1 g/100 kcal, or in an amount from 0.6 to 2.0 g/100 kcal, or in an amount from 0.7 to 2.0 g/100 kcal, or in an amount from 0.8 to 1.8 g/100 kcal, or in an amount from 0.9 to 1.7 g/100 kcal, or in an amount from 1.0 to 1.5 g/100 kcal or in an amount from 1.0 to 1.6 g/100 kcal.

The nutritional composition of the present invention may comprise at least 0.01 wt % of N-acetylated oligosaccharide(s), at least 2.0 wt % of galacto-oligosaccharide(s) and at least 0.02 wt % of sialylated oligosaccharide(s).

In some embodiments, the nutritional composition according to the present invention may comprise at least 0.01 wt %, or at least 0.02 wt %, or at least 0.03 wt %, or at least 0.04 wt %, or at least 0.05 wt %, or at least 0.06 wt % or at least 0.07 wt % of N-acetylated oligosaccharide(s). In some embodiments, it may comprise from 0.01 to 0.07 wt % of N-acetylated oligosaccharide(s) such as from 0.01 to 0.05 wt % of N-acetylated oligosaccharide(s) or from 0.01 to 0.03 wt % of N-acetylated oligosaccharide(s).

In addition, the nutritional composition may comprise at least 2 wt %, or at least 3 wt %, or at least 4 wt %, or at least 5 wt %, or at least 5.5 wt %, or at least 6 wt % or at least 7 wt % or at least 8 wt % of galacto-oligosaccharide(s). In some embodiments, it may comprise from 5 to 8 wt % of galacto-oligosaccharide(s) such as from 5.75 to 7 wt % of galacto-oligosaccharide(s) or from 5.85 to 6.5 wt % of galacto-oligosaccharide(s). A particular example is an amount of 5.95 wt % of oligosaccharide(s).

Finally, the nutritional composition may comprise at least 0.02 wt %, or at least 0.03 wt %, or at least 0.04 wt %, or at least 0.05 wt %, or at least 0.06 wt %, or at least 0.07 wt %, or at least 0.08 wt % or at least 0.09 wt % of sialylated oligosaccharides.

In some embodiments, it may comprise from 0.02 to 0.09 wt % of sialylated oligosaccharide(s) such as from 0.02 to 0.08 wt % of sialylated oligosaccharide(s), or from 0.02 to 0.07 wt % of sialylated oligosaccharide(s) or from 0.003 to 0.07 wt % of sialylated oligosaccharide(s).

In a particular embodiment, the nutritional composition according to the present invention may comprise from 0.01 to 0.07 wt % of N-acetylated oligosaccharide(s), from 2.0 to 8.0 wt % of galacto-oligosaccharide(s) and from 0.02 to 0.09 wt % of sialylated oligosaccharide(s).

In yet another particular embodiment, the nutritional composition according to the present invention may comprise from 0.01 to 0.03 wt % of N-acetylated oligosaccharide(s), 5.95 wt % galacto-oligosaccharide(s) and from 0.02 to 0.09 wt % of sialylated oligosaccharide(s).

In another embodiment, the nutritional composition may comprise at least 0.0015 g/100 kcal of N-acetylated oligosaccharide(s), at least 0.70 g/100 kcal of galacto-oligosaccharide(s) and at least 0.0045 g/100 kcal of sialylated oligosaccharide(s).

In some specific embodiments, the nutritional composition may comprise at least 0.0015 g/100 kcal, or at least 0.002 g/100 kcal, or at least 0.0025 g/100 kcal, or at least 0.003 g/100 kcal, or at least 0.0035 g/100 kcal, or at least 0.004 g/100 kcal, or at least 0.0045 g/100 kcal or at least 0.005 g/100 kcal of N-acetylated oligosaccharide(s). In some embodiments, the nutritional composition may comprise from 0.0015 to 0.005 g/100 kcal of N-acetylated oligosaccharide(s) such as from 0.0015 to 0.045 g/100 kcal of N-acetylated oligosaccharide(s) or from 0.002 to 0.0045 g/100 kcal of N-acetylated oligosaccharide(s).

In addition the nutritional composition may comprise at least 0.70 g/100 kcal, or at least 0.74 g/100 kcal, or at least 0.8 g/100 kcal, or at least 0.85 g/100 kcal, or at least 0.90 g/100 kcal, or at least 0.95 g/100 kcal, or at least 1.0 g/100 kcal, or at least 1.05 g/100 kcal, or at least 1.10 g/100 kcal, or at least 1.20 g/100 kcal or at least 1.50 of galacto-oligosaccharide(s). In some embodiments, it may comprise from 0.70 to 1.5 g/100 kcal of galacto-oligosaccharide(s) such as from 0.70 to 1.20 g/100 kcal of galacto-oligosaccharide(s) or from 0.74 to 1.2 g/100 kcal of galacto-oligosaccharide(s).

Finally the nutritional composition may comprise at least 0.0045 g/100 kcal, or at least 0.005 g/100 kcal, or at least 0.0055 g/100 kcal, or at least 0.006 g/100 kcal, or at least 0.0065 g/100 kcal, or at least 0.007 g/100 kcal, or at least 0.0075 g/100 kcal, or at least 0.008 g/100 kcal or at least 0.0085 g/100 kcal of sialylated oligosaccharide(s). In some embodiments, it may comprise from 0.0045 to 0.0085 g/100 kcal of sialylated oligosaccharide(s) such as from 0.0045 to 0.008 g/100 kcal of sialylated oligosaccharide(s) or from 0.0045 to 0.0075 g/100 kcal of sialylated oligosaccharide(s).

In a particular embodiment, the nutritional composition may comprise from 0.0015 to 0.005 g/100 kcal of N-acetylated oligosaccharide(s), from 0.70 to 1.5 g/100 kcal of galacto-oligosaccharide(s) and from 0.0045 to 0.0085 g/100 kcal of sialylated oligosaccharide(s).

In another particular embodiment, the nutritional composition may comprise from 0.0015 to 0.0045 g/100 kcal of N-acetyl-oligosaccharide(s), from 0.74 to 1.2 g/100 kcal of galacto-oligosaccharide(s) and from 0.0045 to 0.0075 g/100 kcal of sialylated oligosaccharide(s).

In a particular advantageous embodiment, the oligosaccharide mixture of the nutritional composition according to the invention comprises from 0.1 to 4.0 wt % of N-acetylated oligosaccharide(s), from 92.0 to 98.5 wt % of the galacto-oligosaccharide(s) and from 0.3 to 4.0 wt % of the sialylated oligosaccharide(s).

The nutritional composition according to the invention may also contain other types of prebiotic (i.e. different and in addition to the oligosaccharides comprised in the oligosaccharide mixture as defined according to the present invention). A prebiotic is a non-digestible food ingredient that beneficially affects the host by selectively stimulating the growth and/or the activity of one or a limited number of species of bacteria in the colon, and thus improves the host health. Such ingredients are non-digestible in the sense that they are not broken down and absorbed in the stomach or the small intestine and thus pass intact to the colon where they are selectively fermented by the beneficial bacteria. Examples of other types of prebiotics include human milk oligosaccharides (HMOs), oligofructose, fructo-oligosaccharides (FOS), inulin, xylooligosaccharides (XOS), polydextrose or any mixture thereof.

Suitable commercial products that can be used in addition to the oligosaccharides comprised in the oligosaccharide mixture to prepare the nutritional compositions according to the invention include combinations of FOS with inulin such as the product sold by BENEO under the trademark Orafti, or polydextrose sold by Tate & Lyle under the trademark STA-LITE®

The nutritional composition according to the invention can further comprise at least one probiotic (or probiotic strain), such as a probiotic bacterial strain. A probiotic is a microbial cell preparation or components of microbial cells with a beneficial effect on the health of the host. The probiotic microorganisms most commonly used are principally bacteria and yeasts of the following genera: Lactobacillus spp., Streptococcus spp., Enterococcus spp., Bifidobacterium spp. and Saccharomyces spp.

Suitable probiotic strains include Lactobacillus acidophilus, Lactobacillus salivarius, Lactobacillus rhamnosus, Lactobacillus paracasei, Lactobacillus casei, Lactobacillus johnsonii, Lactobacillus plantarum, Lactobacillus fermentum, Lactobacillus lactis, Lactobacillus delbrueckii, Lactobacillus helveticus, Lactobacillus bulgari, Lactococcus lactis, Lactococcus diacetylactis, Lactococcus cremoris, Streptococcus salivarius, Streptococcus thermophilus, Bifidobacterium lactis, Bifidobacterium animalis, Bifidobacterium longum, Bifidobacterium breve, Bifidobacterium infantis, Bifidobacterium adolescentis or any mixture thereof.

Some examples of suitable probiotic bacterial strains include Lactobacillus rhamnosus ATCC 53103 available from Valio Oy of Finland under the trademark LGG, Lactobacillus rhamnosus CGMCC 1.3724, Lactobacillus paracasei CNCM I-2116, Lactobacillus johnsonii CNCM I-1225, Streptococcus salivarius DSM 13084 sold by BLIS Technologies Limited of New Zealand under the designation KI2, Bifidobacterium lactis CNCM I-3446 sold inter alia by the Christian Hansen company of Denmark under the trademark Bb 12, Bifidobacterium longum ATCC BAA-999 sold by Morinaga Milk Industry Co. Ltd. of Japan under the trademark BB536, Bifidobacterium breve sold by Danisco under the trademark Bb-03, Bifidobacterium breve sold by Morinaga under the trade mark M-16V, Bifidobacterium infantis sold by Procter & Gamble Co. under the trademark Bifantis and Bifidobacterium breve sold by Institut Rosell (Lallemand) under the trademark R0070.

The probiotic may be added in an amount between 10e3 and 10e12 cfu/g of composition on a dry weight basis, more preferably between 10e7 and 10e12 cfu/g of composition on a dry weight basis.

The nutritional compositions according to the invention may be for example an infant formula, any other milk-based nutritional composition, a supplements (or a complement), a fortifier such as a milk fortifier. Preferably the nutritional composition according to the invention is an infant formula. An infant formula is formulated with essential nutrients in order to provide a complete nutrition to the infant.

Therefore, the nutritional compositions of the invention, and especially the infant formulas, generally contain a protein source, a carbohydrate source and a lipid source.

The protein source might be based on cow's milk proteins such as whey, casein and mixtures thereof, as well as protein source based on soy. The casein to whey ratio can be in the range of 30:70 to 70:30, such as 40:60, alternatively 45:55 to 50:60, in particular 40:60.

The whey protein may be a whey protein isolate, acid whey, sweet whey or sweet whey from which the caseino-glycomacropeptide has been removed (modified sweet whey). Preferably, however, the whey protein is modified sweet whey. Sweet whey is a readily available by-product of cheese making and is frequently used in the manufacture of nutritional compositions based on cows' milk. However, sweet whey includes a component which is undesirably rich in threonine and poor in tryptophan called caseino-glycomacropeptide (cGMP). Removal of the cGMP from sweet whey results in a protein with a threonine content closer to that of human milk. A process for removing cGMP from sweet whey is described in EP880902.

The protein source generally represents from 1.5 to 3.0 g/100 kcal of the nutritional composition (e.g. infant formula), such as from 1.7 to 2.2 g/100 kcal. It generally contributes between 5-15% of the total energy of the composition.

The protein(s) in the protein source of the nutritional compositions of the invention may be intact or hydrolysed or a combination of intact and hydrolysed proteins. In an embodiment of the invention, the protein(s) in the protein source is hydrolysed. In another embodiment of the invention, the protein(s) in the protein source is intact. The term “intact” means in the context of the present invention proteins where the molecular structure of the protein(s) is not altered according to conventional meaning of intact proteins. By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component peptides or amino acids. The proteins may either be fully or partially hydrolysed. In an embodiment of the invention at least 70% of the proteins are hydrolysed, preferably at least 80% of the proteins are hydrolysed, such as at least 85% of the proteins are hydrolysed, even more preferably at least 90% of the proteins are hydrolysed, such as at least 95% of the proteins are hydrolysed, particularly at least 98% of the proteins are hydrolysed. In a particular embodiment, 100% of the proteins are hydrolysed. Hydrolysis of proteins may be achieved by many means, for example by prolonged boiling in a strong acid or a strong base or by using an enzyme such as the pancreatic protease enzyme to stimulate the naturally occurring hydrolytic process. It may be desirable to add partially hydrolysed proteins (degree of hydrolysation between 2 and 20%), for example for infants believed to be at risk of developing cows' milk allergy.

The carbohydrate source of the nutritional composition (e.g. infant formula), may be lactose, saccharose, maltodextrin, starch and mixture thereof. Advantageously it may be lactose. The carbohydrate source generally represents between 9 and 14 g/100 kcal, such as from 8 to 12 g/100 kcal of the nutritional composition (e.g. infant formula). It generally contributes between 35 and 65% of the total energy of the composition.

The lipid source of the nutritional composition of the invention (e.g. infant formula) may be any lipid or fat which is suitable for use in said compositions. Preferred lipid sources include vegetable fats, such as palm olein, high oleic sunflower oil and high oleic safflower oil, or milk fats. The essential fatty acid linoleic and α-linoleic acid may also be added, as well as small amounts of oil containing high quantities of preformed long chain polyunsaturated fatty acids, such as arachidonic acid and docosahexaenoic acid, e.g. fish oils or microbial oils. In total, the lipid content in the nutritional composition (e.g. infant formula) may be between 3 and 7.5 g/100 kcal, such as from 4.4 to 6 g/100 kcal or from 5 to 7 g/100 kcal. It generally contributes between 30 to 55% of the total energy content of the composition. The fat source has advantageously a ratio of linoleic acid (C18:2n-6) to α-linolenic acid (C18:3n-3) of about 5:1 to about 15:1, for example about 6:1 to about 10:1. It may also have a ratio of arachidonic acid (C20:4n-6) to docosahexaenoic acid (C22:6n-3) between 2:1 and 1:1.

The nutritional composition of the invention (e.g. infant formula) may also contain all vitamins and minerals understood to be essential in the daily diet of the infant and in nutritionally significant amounts. Examples of minerals, vitamins and other nutrients optionally present in the infant formula include vitamin A, vitamin B1, vitamin B2, vitamin B6, vitamin B12, vitamin E, vitamin K, vitamin C, vitamin D, folic acid, inositol, niacin, biotin, panthotenic acid, choline, calcium, sodium, phosphorous, iodine, magnesium, copper, zinc, iron, manganese, chloride, potassium, selenium, chromium, molybdenum, taurine and L-carnitine. Minerals are usually in salt form. The presence and amount of specific minerals and other vitamins may vary depending on the intended infant population.

If necessary, the infant formula will contain emulsifiers and stabilizers such as soy lecithin, citric acid esters of mono- and di-glycerides, and the like.

The nutritional composition of the invention (e.g. infant formula) may optionally contain other substances that may have a beneficial effect such as lactoferrin, nucleotides, nucleosides and the like.

The nutritional composition of the invention (e.g. infant formula) may be prepared by blending together the protein source, the carbohydrate source and the fat source in appropriate proportions. Emulsifiers may be added if desired. Vitamins and minerals may be added at this point but are usually added later to avoid thermal degradation. Any lyophilic vitamins, emulsifiers and the like may be dissolved into the fat source prior to blending. Water, preferably water which has been subjected to reverse osmosis, may then be mixed in to form a liquid mixture.

The liquid mixture may then be thermally treated to reduce bacterial loads. For example, the liquid mixture may be rapidly heated to a temperature in the range of about 80° C. to about 110° C. for about 5 seconds to about 5 minutes. This may be carried out by steam injection or by heat exchanger, e.g. a plate heat exchanger.

The liquid mixture may then by cooled to about 60° C. to about 85° C., for example by flash cooling. The liquid mixture may then be homogenized, for example in two stages at about 7 MPa to about 40 MPa in the first stage and about 2 MPa to about 14 MPa in the second stage. The homogenized mixture may then be further cooled to add any heat sensitive components such as vitamins and minerals. The pH and solids content of the homogenized mixture may be conveniently standardized at this point.

The homogenized mixture may be transferred to a suitable drying apparatus, such as spray drier or freeze drier, and may be converted to powder. The powder should have a moisture content of less than about 5% by weight.

The oligosaccharide mixture may be prepared by any suitable manner known in the art and added at different steps during the preparation of the nutritional composition of the present invention. The oligosaccharide mixture can be added directly to the nutritional composition (e.g. infant formula) by dry mixing (i.e. at the blending step). Alternatively, the oligosaccharide mixture can be added in liquid mixture prior to the thermal treatment to reduce the bacterial load. The individual components of the oligosaccharide mixture may also be added separately to the nutritional composition in which case the oligosaccharide mixture is preferably added in the liquid phase immediately prior to drying.

The nutritional composition of the present invention is administered (or given, fed . . . ) to the infants in the first twelve months of life, i.e. within/during the first twelve months of life of the infants. The nutritional composition can be administered during this entire specific window of time, or during only a part thereof.

For example, the nutritional composition of the invention can be given for some days (1, 2, 3, 4, 5, 6 . . . ), or for some weeks (1, 2, 3, 4, 5, 6, 7, 8 or even more), or for some months (1, 2, 3, 4, 5, 6, 7, 8, 9, 10, 11 or 12) depending on the needs. Therefore in some embodiments the composition of the invention is given during the first week of life of the infant, or during the first 2 weeks of life, or during the first 3 weeks of life, or during the first month of life, or during the first 2 months of life, or during the first 3 months of life, or during the first 4 months of life, or during the first 6 months of life of the infants, or during the first 7 months of life, or during the first 8 months of life, or during the first 9 months of life, or during the first 10 months of life, or during the first 11 months of life, or during the first 12 months of life. In a specific embodiment, the nutritional composition is given during the first months of life of the infant up to 12 months of life. In a particular embodiment, the nutritional composition of the invention is given to the infants within a shorter period of time such as within the first six months of life. In a specific advantageous embodiment, the nutritional composition of the invention is given to the infants within the first month of life. Indeed, without to be bound by the theory, it is believed that providing the nutritional composition within the early age of the infant is particularly efficient.

In some embodiments the nutritional composition of the invention is given immediately after birth.

In some embodiments, the composition of the invention is given few days (1, 2, 3, 4, 5, 6 . . . ), or few weeks (1, 2, 3, 4, 5, 6 . . . ) or few months (1, 2, 3 . . . ) after birth. This may be especially the case when the infant is premature, but not necessarily.

In some other embodiments, it is administered to the infants from (i.e. starting) 2 days of life (i.e. 48 h).

In a specific embodiment, the nutritional composition of the invention is given to the infants from 2 days of life and within the first month of life.

In addition, the administration of the nutritional composition can be continuous or not. In a particular embodiment of the invention, the administration is continuous, i.e. the nutritional composition is fed to the infant at every feed, that is to say at every meal of the infant.

The oligosaccharide mixture present in the nutritional composition of the invention may be prepared from one or more animal milks. The milk can be obtained from any mammal, in particular from cows, goats, buffalos, horses, elephants, camels or sheep. In a specific embodiment, the oligosaccharides of the oligosaccharide mixture are bovine's milk oligosaccharides and can be obtained from cows, goats or buffalos' milk. In an advantageous embodiment, the oligosaccharides are obtained from cow's milk.

In a further aspect, the present invention relates to the use of an oligosaccharide mixture, said oligosaccharide mixture comprising at least one N-acetylated oligosaccharide, one galacto-oligosaccharide and one sialylated oligosaccharide for the preparation of a nutritional composition for use in reducing the metabolic stress (e.g. in reducing the metabolic disorders and/or imbalances) in an infant in the first twelve months of life. It especially encompasses the use of an oligosaccharide mixture, said oligosaccharide mixture comprising at least one N-acetylated oligosaccharide, one galacto-oligosaccharide and one sialylated oligosaccharide for the preparation of a nutritional composition for use in the prevention and/or treatment of conditions and/or diseases associated to the metabolic disorders and/or imbalances in an infant in the first twelve months of life, especially by reducing the metabolic stress in said infant in the first twelve months of life.

In another aspect, the present invention relates to the use of an oligosaccharide mixture, said oligosaccharide mixture comprising at least one N-acetylated oligosaccharide, one galacto-oligosaccharide and one sialylated oligosaccharide for the preparation of a nutritional composition for use in decreasing the gut permeability in an infant in the first twelve months of life.

In still another aspect, the present invention relates to the use of an oligosaccharide mixture, said oligosaccharide mixture comprising at least one N-acetylated oligosaccharide, one galacto-oligosaccharide and one sialylated oligosaccharide for the preparation of a nutritional composition for use in promoting the rate of growth in the first twelve months of life in an infant fed with said nutritional composition which approximates the rate of growth of a breast-fed infant at the same age.

In an additional aspect, the present invention relates to a method for reducing the metabolic stress (e.g. reducing the metabolic disorders and/or imbalances) in an infant during the first twelve months of life, said method comprising administering to said infant a nutritional composition comprising an oligosaccharide mixture, said oligosaccharide mixture comprising at least one N-acetylated oligosaccharide, one galacto-oligosaccharide and one sialylated oligosaccharide. It especially encompasses a method of preventing and/or treating conditions and/or diseases associated to metabolic disorders and/or imbalances in an infant during the first twelve months of life, especially by reducing the metabolic stress in said infant, said method comprising administering to said infant a nutritional composition comprising an oligosaccharide mixture, said oligosaccharide mixture comprising at least one N-acetylated oligosaccharide, one galacto-oligosaccharide and one sialylated oligosaccharide.

In another aspect, the present invention relates to a method for decreasing the gut permeability in an infant during the first twelve months of life, said method comprising administering to said infant a nutritional composition comprising an oligosaccharide mixture, said oligosaccharide mixture comprising at least one N-acetylated oligosaccharide, one galacto-oligosaccharide and one sialylated oligosaccharide.

In still another aspect, the present invention relates to a method for promoting a rate of growth in the first twelve months of life in an infant which approximate the rate growth of an infant breast fed, said method comprising administering to said infant a nutritional composition comprising an oligosaccharide mixture, said oligosaccharide mixture comprising at least one N-acetylated oligosaccharide, one galacto-oligosaccharide and one sialylated oligosaccharide.

The invention will now be illustrated by reference to the following examples.

### Example 1

An example of the composition of an infant formula comprising an oligosaccharide mixture according to the invention is given in the below table 1. The oligosaccharide mixture comprises from 0.1 to 4.0 wt % of the N-acetylated oligosaccharide(s), from 92.0 to 98.5 wt % of the galacto-oligosaccharide(s) and from 0.3 to 4.0 wt % of the sialylated oligosaccharide(s).

TABLE 1

| Nutrient | per 100 kcal | per litre |
|---|---|---|
| Energy (kcal) | 100 | 650 |
| Protein (g) | 2.26 | 14.7 |
| Fat (g) | 5.61 | 36.5 |
| Linoleic acid (g) | 0.65 | 4.2 |
| α-linoleic acid (mg) | 81.2 | 528 |
| Lactose (g) | 10.1 | 65.7 |
| Oligosaccharide Mixture (g) | 1.38 | 9.0 |
| Minerals (g) | 0.38 | 2.5 |
| Na (mg) | 38 | 240 |
| K (mg) | 95 | 620 |
| Cl (mg) | 80 | 520 |
| Ca (mg) | 60 | 390 |
| P (mg) | 33 | 220 |
| Mg (mg) | 7 | 45.5 |
| Mn (μg) | 5 | 30 |
| Se (μg) | 4.0 | 26 |
| Vitamin A (μg RE) | 110 | 730 |
| Vitamin D (μg RE) | 1.5 | 10 |
| Vitamin E (mg TE) | 1.3 | 8.7 |
| Vitamin K1 (μg) | 8 | 52 |
| Vitamin C (mg) | 15 | 98 |
| Vitamin B1 (mg) | 0.07 | 0.46 |
| Vitamin B2 (mg) | 0.10 | 0.65 |
| Niacin (mg) | 0.5 | 3.3 |
| Vitamin B6 (mg) | 0.049 | 0.32 |
| Folic acid (μg) | 16 | 100 |
| Pantothenic acid (mg) | 0.7 | 4.6 |
| Vitamin B12 (μg) | 0.2 | 1.3 |
| Biotin (μg) | 2.0 | 13 |
| Choline (mg) | 20 | 130 |
| Fe (mg) | 0.71 | 4.6 |
| I (μg) | 20 | 130 |
| Cu (mg) | 0.08 | 0.52 |
| Zn (mg) | 1.2 | 7.8 |

### Example 2

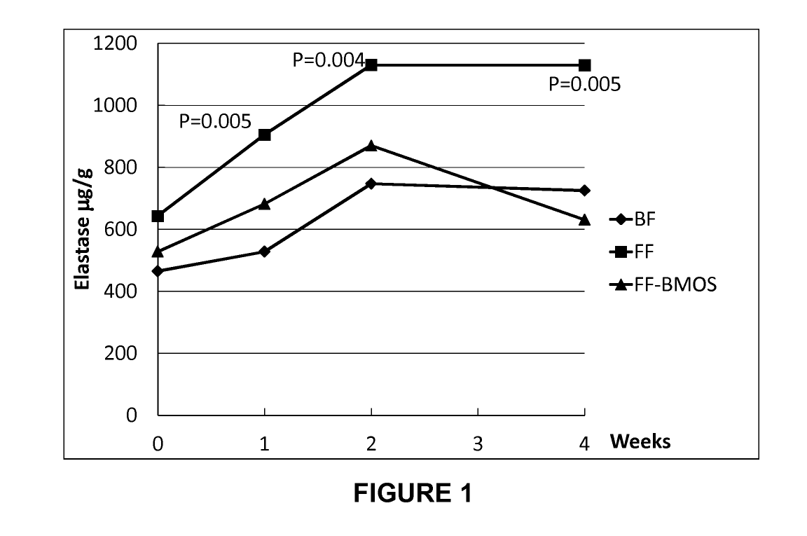

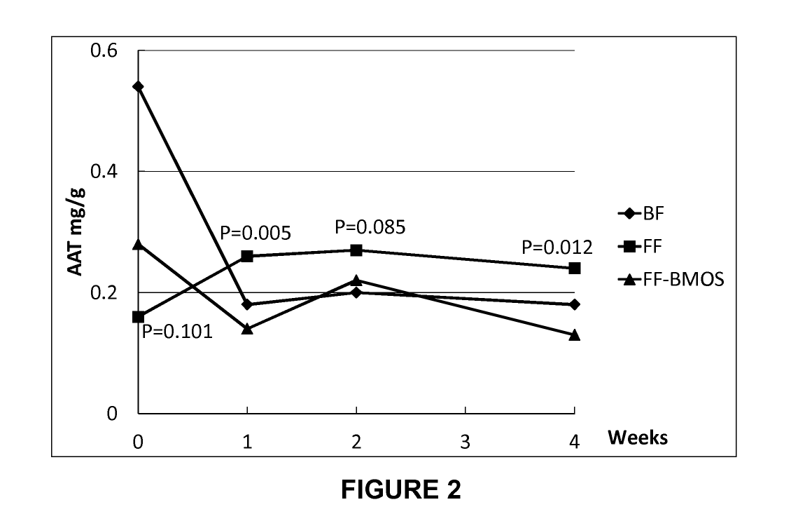

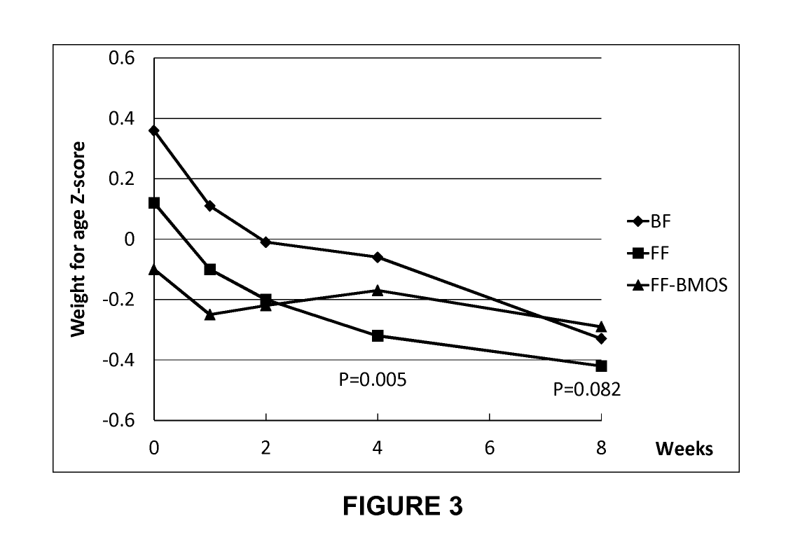

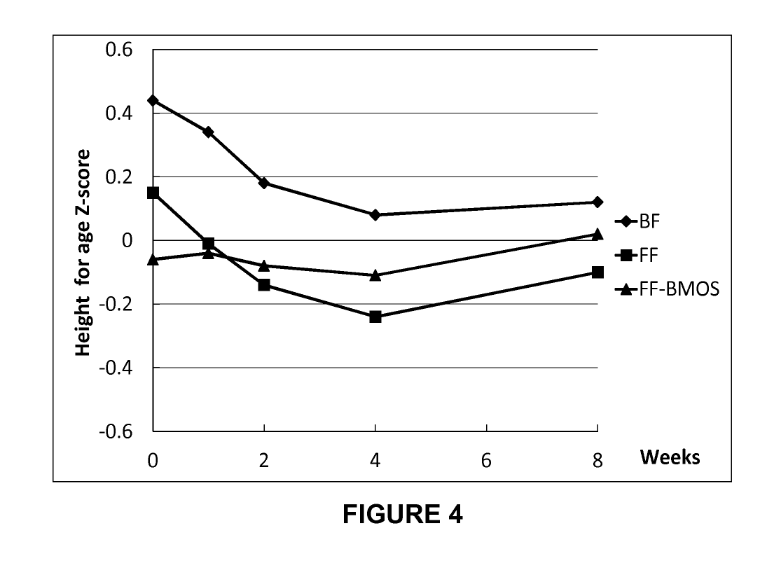

The present example illustrates the effects of a nutritional composition as indicated in example 1 for reducing the metabolic stress (FIG. 1 and Table 2), for decreasing the gut permeability (FIG. 2 and Table 3) and for promoting the growth rate of infant fed with the nutritional composition (FIGS. 3 and 4).

### 2.1 Experimental Design

This was a multi-centres, parallel-group, double-blind, randomized controlled trial. A total of 202 healthy term new-borns were recruited from mothers attending the study centres for perinatal care. Infants from mothers who had chosen not to breast-feed beyond 48 hours following birth were enrolled into formula groups whereas a reference group of breast-fed (BF) infants were recruited from mothers who intended to breast-feed for at least 4 months starting from the infant's birth.

From randomization (first 48 hours of life) to 1 month of age infants received a standard infant formula (FF) or the same standard infant formula but supplemented with bovine milk oligosaccharides obtained from cow's milk (FF+BMOs). Then from 1 to 2 months of age, infant formula was given to all formula fed infants. 127 formula fed infants (FF:44 and FF+BMOs:43) and 75 breast-fed infants were enrolled. The study formulas contained sufficient amounts of proteins, carbohydrates, fats, vitamins and minerals for normal growth of infants from birth to 6 months of age. All of the formulas also contained long chain polyunsaturated fatty acids and provided 65 kcal/100 ml of reconstituted formula. Both, the infant formula as the infant formula supplemented with BMOs (both developed and produced at Nestlé Product Technology Center, Konolfingen, Switzerland) were similar in composition except that the latter contains BMOs at a total concentration (i.e total oligosaccharide mixture) of 7.5±1 g/100 g of powder formula (1.38±0.2 g/100 kcal).

### 2.2 Sample Collection and Analytical Measures

### 2.2.1 Anthropometric Measures and Sample Collection

Follow-up visits to the study centre took place at 7 days (1 week), 14 days (2 weeks) and 28 days (4 weeks) of age.

Anthropometric measures were taken during each of these visits, infants were weighted nude to the nearest 10 g on the same electronic scales calibrated according to the manufacturer's specifications and recumbent length was measured to the nearest 10 mm with the full body extended and feet flexed.

5 g of fresh stool were collected at birth (0), 1 week, 2 weeks and 4 weeks of age from infants of the three groups at the study site and either used directly for analysis or stored for further analysis.

### 2.2.2 Elastase and AAT Measures

1 g of stool sample was mixed with 5 ml of extraction buffer containing a Protease Inhibitor cocktail (purchased at Roche Applied Bioscience) dissolved in Dubelcco's Phosphate Buffered Saline (purchased from SIGMA). Samples were centrifuged and supernatants were analysed for levels of Elastase (Kit ELISA Schebo Biotech, Elastase 1 pancréatique, Ref. 07, DE) or AAT (Kit ELISA for a-1-antitrypsin (Immundiagnostik, DE).

### 2.3 Results

### 2.3.1 Effect of a Nutritional Composition Comprising the Oligosaccharide Mixture According to the Present Invention on Reducing Metabolic Stress in Infants

The effect of a nutritional composition of the present invention on the metabolic stress was monitored by measuring the level of fecal Elastase in the group of infants breast-fed (BF), the group of infants fed with a standard infant formula (FF) and the group of infants fed with the standard infant formula comprising the oligosaccharide mixture according to the present invention (FF+BMOS).

FIG. 1 and Table 2 show that feeding the infants with a nutritional composition according to the invention (FF+BMOS) reduced significantly the levels of fecal Elastase within the first four weeks of life, as compared to the levels of fecal Elastase measured in the FF group. In addition, the levels of fecal Elastase measured in the FF+BMOS group are close to the levels of fecal Elastase measured in the BF control group. On the contrary, levels of fecal Elastase measured in the FF group were significantly higher than the levels of the BF control group and of the FF+BMOS group. Elevated levels of fecal Elastase correlate with increased hepatic activity and therefore is indicating metabolic stress. Considering the level of fecal Elastase measured in the BF group as the reference metabolic activity, the nutritional composition according to the invention clearly reduces the metabolic stress towards normal metabolic activity as observed in the breast-fed infants. Altogether, these results indicate that the metabolic activity is buffered by the addition of BMOs in a standard nutritional composition (e.g. a standard infant formula) and matches the pattern obtained in BF group.

TABLE 2
Fecal Elastase levels (μg/g)

| | BF | FF | FF + BMOs |
|---|---|---|---|
| Weeks | n = 75 | n = 44 | n = 43 |
| 0 | 465 | 643 | 528 |
| 1 | 528 | 905 | 682 |
| 2 | 747 | 1130 | 870 |
| 4 | 725 | 1129 | 631 |

### 2.3.2 Effect of a Nutritional Composition Comprising the Oligosaccharide Mixture According to the Present Invention on Decreasing the Gut Permeability in Infants

The effect of the nutritional composition of the present invention on the gut permeability was monitored by measuring the level of fecal α1-antitrypsin (AAT) in the group of infants breast-fed (BF), the group of infants fed with an infant formula (FF) and the group of infants fed with the nutritional composition comprising an oligosaccharide mixture (FF+BMOS).

FIG. 2 and Table 3 show that feeding the infants with the nutritional composition according to the invention (FF+BMOS) reduced significantly the levels of AAT within the first four weeks of life, as compared to the level of fecal AAT measured in the FF group. More importantly, the levels of fecal AAT measured in the FF+BMOS group approximate the levels of fecal AAT measured in the BF group. As the levels of fecal AAT measured in the infant fed with the nutritional composition of the present invention approach the levels of fecal AAT measured in the breast-fed infants, it may mitigate the risk of increased inflammatory response.

Altogether, these results indicate that a nutritional composition comprising the oligosaccharide mixture according to the present invention protects the intestinal barrier function and therefore decreases the gut permeability.

TABLE 3
Fecal AAT levels (mg/g)

| | BF | FF | FF + BMOs |
|---|---|---|---|
| Weeks | n = 75 | n = 44 | n = 43 |
| 0 | 0.54 | 0.16 | 0.28 |
| 1 | 0.18 | 0.26 | 0.14 |
| 2 | 0.2 | 0.27 | 0.22 |
| 4 | 0.18 | 0.24 | 0.13 |

### 2.3.3 Effect of a Nutritional Composition Comprising an Oligosaccharide Mixture According to the Present Invention on the Growth of Infants

The growth of infants of the different groups (FF, FF+BMOS and BF) was monitored over 8 weeks by measuring two recognized growth parameters: weight (FIG. 3 and Table 4) and height (FIG. 4 and Table 4). The measures were plotted against the standard statistical values provided by the WHO (i.e. Z-score). Any significant deviation from the Z-score may indicate an abnormal growth.

As illustrated in FIG. 3 and Table 4, the weight for age Z-score was significantly higher in the group of infants fed with the nutritional composition of the present invention (FF+BMOS) as compared to the group of infants fed with the standard infant formula (FF). Importantly, the weight for age Z-score of the FF+BMOS group is closer to the group of infant breast-fed (BF). This strong trend can be observed over 8 weeks, meaning that it persisted during the follow-up period of the study (i.e. 4 weeks after the stop of feeding with FF+BMOS).

As illustrated in FIG. 4 and Table 4, the height for age Z-score was also higher in the group of infants fed with the nutritional composition of the present invention (FF+BMOS) as compared to the groups fed with the standard formula (FF). Surprisingly, the height for age Z-score for the group FF+BMOS was in the range of the WHO standard value (height for age Z-score=0).

Altogether, these measures indicate that a nutritional composition comprising the oligosaccharide mixture according to the present invention promotes a growth rate of the infants which approximates the growth rate of breast-fed infants at the same age, and therefore approaches the standard growth rate values used to define a healthy growth.

TABLE 4
Anthropometric measures (weight and height) (Z-score)

| | BF | | FF | | FF + BMOs | |
|---|---|---|---|---|---|---|
| | n = 75 | | n = 45 | | n = 43 | |
| Weeks | Weight | Height | Weight | Height | Weight | Height |
| 0 | 0.36 | 0.44 | 0.12 | 0.15 | −0.1 | −0.06 |
| 1 | 0.11 | 0.34 | −0.1 | −0.01 | −0.25 | −0.04 |
| 2 | −0.01 | 0.18 | −0.2 | −0.14 | −0.22 | −0.08 |
| 4 | −0.06 | 0.08 | −0.32 | −0.24 | −0.17 | −0.11 |
| 8 | −0.33 | 0.12 | −0.42 | −0.1 | −0.29 | 0.02 |

1. A method for reducing the metabolic stress in an infant in the first twelve months of life comprising administering to the infant a nutritional composition comprising an oligosaccharide mixture, the oligosaccharide mixture comprising at least one N-acetylated oligosaccharide, one galacto-oligosaccharide and one sialylated oligosaccharide.

2. A method for decreasing the gut permeability in an infant in the first twelve months of life comprising administering to the infant a nutritional composition comprising an oligosaccharide mixture, the oligosaccharide mixture comprising at least one N-acetylated oligosaccharide, one galacto-oligosaccharide and one sialylated oligosaccharide.

3. A method for promoting a rate of growth in the first twelve months of life in an infant fed with said nutritional composition which approximates to the rate of growth of a breast-fed infant at the same age comprising administering to the infant a nutritional composition comprising an oligosaccharide mixture, the oligosaccharide mixture comprising at least one N-acetylated oligosaccharide, one galacto-oligosaccharide and one sialylated oligosaccharide.

4. The method of claim 1, wherein the N-acetylated oligosaccharide is selected from the group consisting of GalNAcα1,3Galβ1,4Glc, Galβ1,6GalNAcα1,3Galβ1,4Glc and mixtures thereof.

5. The method according to claim 1, wherein the galacto-oligosaccharide is selected from the group consisting of Galβ1,3Galβ1,4Glc, Galβ1,6Galβ1,4Glc, Galβ1,3Galβ1,3Galβ1,4Glc, Galβ1,6Galβ1,6Galβ1,4Glc, Galβ1,3Galβ1,6Galβ1,4Glc, Galβ1,6Galβ1,3Galβ1,4Glc, Galβ1,6Galβ1,6Galβ1,6Glc, Galβ1,3Galβ1,3Glc, Galβ1,4Galβ1,4Glc, Galβ1,4Galβ1,4Galβ1,4Glc and mixtures thereof.

6. The method according to claim 1, wherein the sialylated oligosaccharide is selected from the group consisting of NeuAcα2,3Galβ1,4Glc, NeuAcα2,6Galβ1,4Glc and mixtures thereof.

7. The nutritional composition according to claim 1 wherein the oligosaccharide mixture is present in an amount of from 2.5 to 15.0 wt %.

8. The nutritional composition according to claim 1 comprising at least 0.01 wt % of N-acetylated oligosaccharide(s), at least 2.0 wt % of galacto-oligosaccharide(s) and at least 0.02 wt % of sialylated oligosaccharide(s).

9. The method according to claim 1 wherein oligosaccharide mixture comprises from 0.1 to 4.0 wt % of the N-acetylated oligosaccharide(s), from 92.0 to 98.5 wt % of the galacto-oligosaccharide(s) and from 0.3 to 4.0 wt % of the sialylated oligosaccharide(s).

10. The method according to claim 1 wherein the composition comprises a prebiotic, selected from the group consisting of human milk oligosaccharides, fructo-oligosaccharide, inulin, xylooligosaccharides, polydextrose and combinations thereof.

11. The method according to claim 1 wherein the composition comprises a probiotic.

12. The method of claim 11, wherein the probiotic is a probiotic bacterial strain selected from the group consisting of Lactobacillus acidophilus, Lactobacillus salivarius, Lactobacillus rhamnosus, Lactobacillus paracasei, Lactobacillus casei, Lactobacillus johnsonii, Lactobacillus plantarum, Lactobacillus fermentum, Lactobacillus lactis, Lactobacillus delbrueckii, Lactobacillus helveticus, Lactobacillus bulgari, Lactococcus lactis, Lactococcus diacetylactis, Lactococcus cremoris, Streptococcus salivarius, Streptococcus thermophilus, Bifidobacterium lactis, Bifidobacterium animalis, Bifidobacterium longum, Bifidobacterium breve, Bifidobacterium infantis, or Bifidobacterium adolescentis and mixtures thereof.

13. The method according to claim 1 wherein the composition is an infant formula.

14. The method according to claim 1 wherein the composition is administered to the infant within the first six months of life.

15. The method according to claim 1 wherein the composition is administered to the infant within the first month of life.

16. The method according to claim 1 wherein the oligosaccharide mixture is derived from animal milk.

17. The method of claim 16 wherein the oligosaccharide mixture is derived from one or more cow's milk, goat's milk and buffalo's milk.

18. The method of claim 2, wherein the N-acetylated oligosaccharide is selected from the group consisting of GalNAcα1,3Galβ1,4Glc, Galβ1,6GalNAcα1,3Galβ1,4Glc and mixtures thereof.

19. The method according to claim 2, wherein the galacto-oligosaccharide is selected from the group consisting of Galβ1,3Galβ1,4Glc, Galβ1,6Galβ1,4Glc, Galβ1,3Galβ1,3Galβ1,4Glc, Galβ1,6Galβ1,6Galβ1,4Glc, Galβ1,3Galβ1,6Galβ1,4Glc, Galβ1,6Galβ1,3Galβ1,4Glc, Galβ1,6Galβ1,6Galβ1,6Glc, Galβ1,3Galβ1,3Glc, Galβ1,4Galβ1,4Glc, Galβ1,4Galβ1,4Galβ1,4Glc and mixtures thereof.

20. The method according to claim 2, wherein the sialylated oligosaccharide is selected from the group consisting of NeuAcα2,3Galβ1,4Glc, NeuAcα2,6Galβ1,4Glc and mixtures thereof.

21. The nutritional composition according to claim 2 wherein the oligosaccharide mixture is present in an amount of from 2.5 to 15.0 wt %.

22. The nutritional composition according to claim 2 comprising at least 0.01 wt % of N-acetylated oligosaccharide(s), at least 2.0 wt % of galacto-oligosaccharide(s) and at least 0.02 wt % of sialylated oligosaccharide(s).

23. The method according to claim 2 wherein oligosaccharide mixture comprises from 0.1 to 4.0 wt % of the N-acetylated oligosaccharide(s), from 92.0 to 98.5 wt % of the galacto-oligosaccharide(s) and from 0.3 to 4.0 wt % of the sialylated oligosaccharide(s).

24. The method according to claim 2 wherein the composition comprises a prebiotic, selected from the group consisting of human milk oligosaccharides, fructo-oligosaccharide, inulin, xylooligosaccharides, polydextrose and combinations thereof.

25. The method according to claim 2 wherein the composition comprises a probiotic.

26. The method of claim 25, wherein the probiotic is a probiotic bacterial strain selected from the group consisting of Lactobacillus acidophilus, Lactobacillus salivarius, Lactobacillus rhamnosus, Lactobacillus paracasei, Lactobacillus casei, Lactobacillus johnsonii, Lactobacillus plantarum, Lactobacillus fermentum, Lactobacillus lactis, Lactobacillus delbrueckii, Lactobacillus helveticus, Lactobacillus bulgari, Lactococcus lactis, Lactococcus diacetylactis, Lactococcus cremoris, Streptococcus salivarius, Streptococcus thermophilus, Bifidobacterium lactis, Bifidobacterium animalis, Bifidobacterium longum, Bifidobacterium breve, Bifidobacterium infantis, or Bifidobacterium adolescentis and mixtures thereof.

27. The method according to claim 2 wherein the composition is an infant formula.

28. The method according to claim 2 wherein the composition is administered to the infant within the first six months of life.

29. The method according to claim 2 wherein the composition is administered to the infant within the first month of life.

30. The method according to claim 2 wherein the oligosaccharide mixture is derived from animal milk.

31. The method of claim 30 wherein the oligosaccharide mixture is derived from one or more cow's milk, goat's milk and buffalo's milk.

32. The method of claim 3, wherein the N-acetylated oligosaccharide is selected from the group consisting of GalNAcα1,3Galβ1,4Glc, Galβ1,6GalNAcα1,3Galβ1,4Glc and mixtures thereof.

33. The method according to claim 3, wherein the galacto-oligosaccharide is selected from the group consisting of Galβ1,3Galβ1,4Glc, Galβ1,6Galβ1,4Glc, Galβ1,3Galβ1,3Galβ1,4Glc, Galβ1,6Galβ1,6Galβ1,4Glc, Galβ1,3Galβ1,6Galβ1,4Glc, Galβ1,6Galβ1,3Galβ1,4Glc, Galβ1,6Galβ1,6Galβ1,6Glc, Galβ1,3Galβ1,3Glc, Galβ1,4Galβ1,4Glc, Galβ1,4Galβ1,4Galβ1,4Glc and mixtures thereof.

34. The method according to claim 3, wherein the sialylated oligosaccharide is selected from the group consisting of NeuAcα2,3Galβ1,4Glc, NeuAcα2,6Galβ1,4Glc and mixtures thereof.

35. The nutritional composition according to claim 3 wherein the oligosaccharide mixture is present in an amount of from 2.5 to 15.0 wt %.

36. The nutritional composition according to claim 3 comprising at least 0.01 wt % of N-acetylated oligosaccharide(s), at least 2.0 wt % of galacto-oligosaccharide(s) and at least 0.02 wt % of sialylated oligosaccharide(s).

37. The method according to claim 3 wherein oligosaccharide mixture comprises from 0.1 to 4.0 wt % of the N-acetylated oligosaccharide(s), from 92.0 to 98.5 wt % of the galacto-oligosaccharide(s) and from 0.3 to 4.0 wt % of the sialylated oligosaccharide(s).

38. The method according to claim 3 wherein the composition comprises a prebiotic, selected from the group consisting of human milk oligosaccharides, fructo-oligosaccharide, inulin, xylooligosaccharides, polydextrose and combinations thereof.

39. The method according to claim 3 wherein the composition comprises a probiotic.

40. The method of claim 39, wherein the probiotic is a probiotic bacterial strain selected from the group consisting of Lactobacillus acidophilus, Lactobacillus salivarius, Lactobacillus rhamnosus, Lactobacillus paracasei, Lactobacillus casei, Lactobacillus johnsonii, Lactobacillus plantarum, Lactobacillus fermentum, Lactobacillus lactis, Lactobacillus delbrueckii, Lactobacillus helveticus, Lactobacillus bulgari, Lactococcus lactis, Lactococcus diacetylactis, Lactococcus cremoris, Streptococcus salivarius, Streptococcus thermophilus, Bifidobacterium lactis, Bifidobacterium animalis, Bifidobacterium longum, Bifidobacterium breve, Bifidobacterium infantis, or Bifidobacterium adolescentis and mixtures thereof.

41. The method according to claim 3 wherein the composition is an infant formula.

42. The method according to claim 3 wherein the composition is administered to the infant within the first six months of life.

43. The method according to claim 3 wherein the composition is administered to the infant within the first month of life.

44. The method according to claim 3 wherein the oligosaccharide mixture is derived from animal milk.

45. The method of claim 44 wherein the oligosaccharide mixture is derived from one or more cow's milk, goat's milk and buffalo's milk.
